(19) United States
(12) Patent Application Publication
(10) Pub. No.: US 2022/0340542 A1
(43) Pub. Date: Oct. 27, 2022

(54) SALTS OF OMECAMTIV MECARBIL AND SOLID FORMS THEREOF

(71) Applicant: SYNTHON B.V., Nijmegen (NL)

(72) Inventors: Katerina JELINKOVA, Blansko (CZ); Michal HEGEDUS, Blansko (CZ)

(21) Appl. No.: 17/761,944

(22) PCT Filed: Sep. 18, 2020
(86) PCT No.: PCT/EP2020/076180

§ 371 Date: Mar. 18, 2022

(30) Foreign Application Priority Data
Sep. 19, 2019 (EP) 19198452.5
Mar. 19, 2020 (EP) 20164365.7

(51) Int. Cl. C07D 401/12

(52) U.S. Cl. CPC C07D 401/12; C07B 2200/13

(57) ABSTRACT

The presented invention relates to Omecamtiv mecarbil salts, solid forms thereof and to processes for preparation thereof.

### BACKGROUND OF THE PRESENT INVENTION

The invention relates to salts of Omecamtiv mecarbil and solid forms thereof. This invention relates to salts of Omecamtiv mecarbil, compound of formula (1), solid forms thereof and processes for preparation thereof;

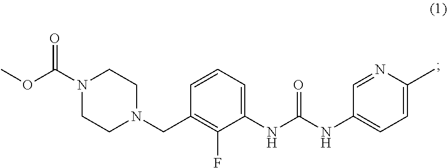

Omecamtiv mecarbil, 4-[2-Fluoro-3-[3-(6-methylpyridin-3-yl)ureido]benzyl]piperazine-1-carboxylic acid methyl ester, a small-molecule activator of cardiac myosin. Omecamtiv mecarbil is in phase III of clinical development for the oral treatment of chronic heart failure.

Omecamtiv mecarbil was disclosed in WO2006009726 application. WO2014152270 application discloses Omecamtiv mecarbil dihydrochloride salt, monohydrate thereof and solid forms thereof.

Modified release formulation of Omecamtiv mecarbil dihydrochloride was developed with the goal of preserving overall bioavailability while lowering Cₘₐₓ (maximum plasma concentration) and the peak-to-trough fluctuation at steady state. That was achieved by using a controlled released pharmaceutical composition comprising release modifying excipients. The same result can be achieved by using omecamtiv mecarbil salt having lower solubility than prior art dihydrochloride salt. It is therefore advantageous to develop Omecamtiv mecarbil salts having low solubility.

### BRIEF DESCRIPTION OF THE INVENTION

The presented invention relates to Omecamtiv mecarbil maleic acid salt, a solid form thereof and a process for preparation thereof.

The presented invention relates to Omecamtiv mecarbil fumaric acid (1:2) salt, a solid form thereof and a process for preparation thereof.

The presented invention further relates to Omecamtiv mecarbil fumaric acid (1:1) salt, solid forms thereof and processes for preparation thereof.

The presented invention also relates to Omecamtiv mecarbil malonic acid (1:1) salt, a solid form thereof and a process for preparation thereof.

The presented invention also relates to Omecamtiv mecarbil adipic acid (1:1.5) salt, a solid form thereof and a process for preparation thereof.

The presented invention further relates to Omecamtiv mecarbil sulfuric acid salt, solid forms thereof and a process for preparation thereof.

The presented invention further relates to a pharmaceutical composition comprising Omecamtiv mecarbil salt of the presented invention.

Omecamtiv mecarbil salt of the presented invention shows lower solubility than prior art dihydrochloride salt, good crystallinity, purity and stability.

### BRIEF DESCRIPTION OF THE DRAWINGS

FIG. 1 depicts the X-Ray Powder Diffractogram (XRPD) of Omecamtiv mecarbil maleic acid salt, Form A, prepared according to Example 1.

FIG. 2 depicts the DSC pattern of Omecamtiv mecarbil maleic acid salt, Form A, prepared according to Example 1.

FIG. 3 depicts NMR spectrum of Omecamtiv mecarbil maleic acid salt, Form A, prepared according to Example 1.

FIG. 4 depicts the X-Ray Powder Diffractogram (XRPD) of Omecamtiv mecarbil fumaric acid (1:2) salt, Form I, prepared according to Example 2.

FIG. 5 depicts the DSC pattern of Omecamtiv mecarbil fumaric acid (1:2) salt, Form I, prepared according to Example 2.

FIG. 6 depicts the NMR spectrum of Omecamtiv mecarbil fumaric acid (1:2) salt, Form I, prepared according to Example 6.

FIG. 7 depicts the X-Ray Powder Diffractogram (XRPD) of Omecamtiv mecarbil fumaric acid (1:1) salt, Form I, prepared according to Example 3.

FIG. 8 depicts the DSC pattern of Omecamtiv mecarbil fumaric acid (1:1) salt, Form I, prepared according to Example 3.

FIG. 9 depicts the NMR spectrum of Omecamtiv mecarbil fumaric acid (1:1) salt, Form I, prepared according to Example 3.

FIG. 10 depicts the X-Ray Powder Diffractogram (XRPD) of Omecamtiv mecarbil fumaric acid (1:1) salt, Form II.

FIG. 11 depicts the X-Ray Powder Diffractogram (XRPD) of Omecamtiv mecarbil malonic acid (1:1) salt, Form I, prepared according to Example 4.

FIG. 12 depicts the DSC pattern of Omecamtiv mecarbil malonic acid (1:1) salt, Form I, prepared according to Example 4.

FIG. 13 depicts the NMR spectrum of Omecamtiv mecarbil malonic acid (1:1) salt, Form I, prepared according to Example 4.

FIG. 14 depicts the X-Ray Powder Diffractogram (XRPD) of Omecamtiv mecarbil adipic acid (1:1.5) salt, Form I, prepared according to Example 5.

FIG. 15 depicts the DSC pattern of Omecamtiv mecarbil adipic acid (1:1.5) salt, Form I, prepared according to Example 5.

FIG. 16 depicts the NMR spectrum of Omecamtiv mecarbil adipic acid (1:1.5) salt, Form I, prepared according to Example 5.

FIG. 17 depicts the X-Ray Powder Diffractogram (XRPD) of Omecamtiv mecarbil sulfuric acid salt, Form I, prepared according to Example 6.

FIG. 18 depicts the DSC pattern of Omecamtiv mecarbil sulfuric acid salt, Form I, prepared according to Example 6.

FIG. 19 depicts the X-Ray Powder Diffractogram (XRPD) of Omecamtiv mecarbil sulfuric acid salt, Form II, prepared according to Example 7.

FIG. 20 depicts the DSC pattern of Omecamtiv mecarbil sulfuric acid salt, Form II, prepared according to Example 7.

FIG. 21 depicts the X-Ray Powder Diffractogram (XRPD) of Omecamtiv mecarbil sulfuric acid salt, Form III.

FIG. 22 depicts the DSC pattern of Omecamtiv mecarbil fumaric acid (1:1) salt, Form II.

### DETAILED DESCRIPTION OF THE INVENTION

The presented invention relates to Omecamtiv mecarbil maleic acid salt, a solid form thereof and a process for preparation thereof. The solid form, Form A, can be characterized by XRPD pattern having 2θ values 10.0°, 15.30 and 16.1° 2 theta (±0.2 degrees 2 theta). The solid Form A can be also characterized by XRPD pattern having 2θ values 10.0°, 10.5°, 13.30, 15.30 and 16.10 degrees 2 theta (f 0.2 degrees 2 theta). The solid form can be further characterized by XRPD pattern described in the following table:

| Angle 2-Theta ° | Intensity % |
|---|---|
| 5.0 | 6.3 |
| 6.6 | 3.6 |
| 7.2 | 3.2 |
| 9.2 | 2.2 |
| 9.6 | 3.3 |
| 10.0 | 100.0 |
| 10.5 | 20.6 |
| 13.0 | 3.5 |
| 13.3 | 17.2 |
| 14.6 | 4.9 |
| 14.9 | 8.8 |
| 15.3 | 64.3 |
| 15.6 | 21.2 |
| 16.1 | 50.1 |
| 16.4 | 17.7 |
| 17.1 | 6.3 |
| 17.5 | 27.0 |
| 17.7 | 22.7 |
| 18.2 | 9.4 |
| 18.5 | 3.5 |
| 19.0 | 17.4 |
| 19.3 | 5.1 |
| 20.2 | 15.0 |
| 20.7 | 3.0 |
| 21.5 | 27.5 |
| 21.8 | 14.3 |
| 22.1 | 8.0 |
| 22.4 | 9.5 |
| 23.1 | 4.7 |
| 23.6 | 3.3 |
| 24.4 | 9.3 |
| 24.7 | 8.5 |
| 25.7 | 8.6 |
| 26.1 | 3.9 |
| 26.7 | 11.5 |
| 27.0 | 55.2 |
| 27.9 | 8.1 |
| 28.7 | 9.6 |
| 29.3 | 2.3 |
| 30.4 | 2.9 |
| 30.8 | 4.7 |
| 31.4 | 2.3 |
| 31.7 | 4.8 |
| 32.1 | 4.4 |
| 32.3 | 5.3 |
| 32.7 | 5.0 |
| 33.1 | 4.0 |
| 33.7 | 4.2 |
| 34.3 | 4.4 |
| 34.5 | 5.7 |

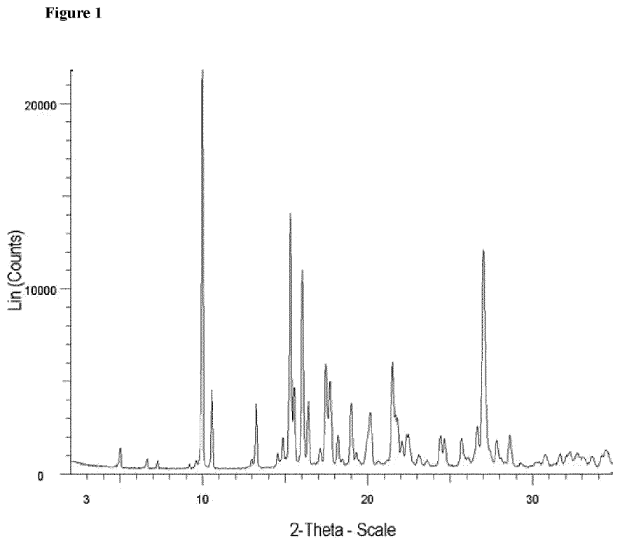

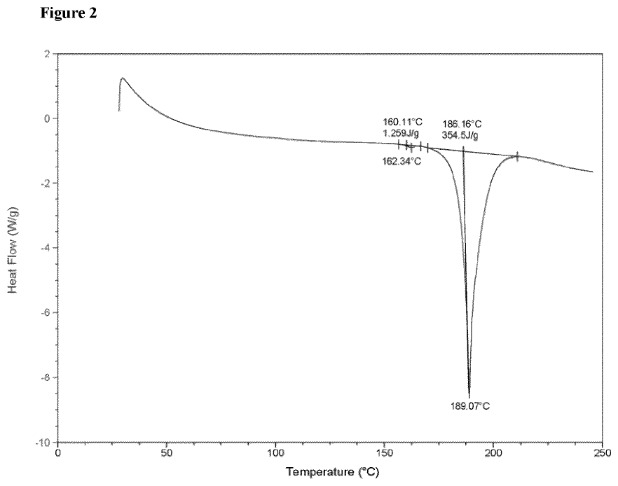

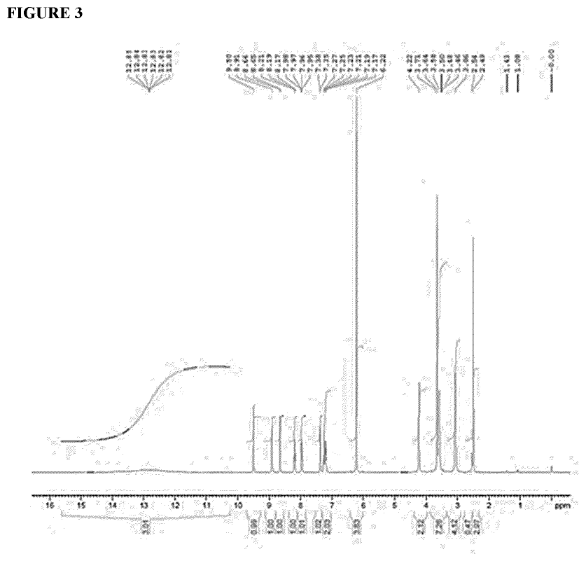

The solid Form A can be further characterized by XRPD pattern depicted in FIG. 1, DSC pattern depicted in FIG. 2 and NMR spectrum depicted in FIG. 3. The solid form of Omecamtiv mecarbil maleic acid salt can be prepared by a process comprising:
a. Dissolving of Omecamtiv mecarbil in an alcohol or acetone or 2-butanone or tetrahydrofurane;
b. Adding of maleic acid;
c. Isolating the solid form.

The alcohol is step a. can be selected from methanol or ethanol or propanol or isopropanol or 1-butanol or 2-butanol. The concentration of Omecamtiv mecarbil in the solvent can be:
1. In case the solvent is an alcohol between 0.04 g/ml and 0.11 g/ml, preferably it is between 0.07 g/ml and 0.11 g/ml;
2. In case the solvent is acetone between 0.007 g/ml and 0.02 g/ml, preferably between 0.009 g/ml and 0.012 g/ml;
3. In case the solvent is 2-butanone between 0.008 g/ml and 0.05 g/ml, preferably between 0.03 g/ml and 0.05 g/ml;
4. In case the solvent is tetrahydrofurane between 0.008 g/ml and 0.04 g/ml, preferably between 0.02 g/ml and 0.04 g/ml.

Omecamtiv mecarbil can be dissolved at an elevated temperature for example between 50° C. and 120° C., preferably between 50° C. and 100° C. To the solution maleic acid is added. Maleic acid can be added as solid or in form of a solution in used solvent. In case maleic acid is used as a solution the concentration of the solution can be between 0.07 and 0.2 g/ml. The molar ration between Omecamtiv mecarbil and maleic acid can be between 1:1 and 1:1.3, preferably it is 1:1.

The mixture is cooled to a temperature between −30° C. and 40° C., preferably between 0° C. and 30° C., more preferably between 15° C. and 25° C. and stirred for between 5 and 20 hours. The solid form can be isolated by any suitable technique, for example using filtration or centrifuge.

A preparation of salts of Omecamtiv mecarbil with acetic acid or benzoic acid was tested using above disclosed process. Only free form of Omecamtiv mecarbil was obtained.

The presented invention also relates to Omecamtiv mecarbil fumaric acid (1:2) salt, a solid form (Form I) thereof and a process for preparation thereof.

The solid form, Form I can be characterized by XRPD pattern having 2θ values 7.6°, 11.7°, 13.7° and 27.8° degrees 2 theta (f 0.2 degrees 2 theta). The solid Form I can be also characterized by XRPD pattern having 2θ values 7.6°, 11.2°, 11.7°, 13.7°, 16.4°, 20.60, 23.50 and 27.8° degrees 2 theta (±0.2 degrees 2 theta). The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 3.7 | 44.6 | 15.0 | 37.4 | 23.5 | 79.4 |
| 7.6 | 75.8 | 15.2 | 43.2 | 24.4 | 23.4 |
| 11.2 | 42.7 | 16.4 | 57.5 | 25.8 | 25.8 |
| 11.7 | 81.6 | 17.2 | 27.6 | 27.0 | 40.6 |
| 12.8 | 44.2 | 18.8 | 33.1 | 27.8 | 100.0 |
| 13.2 | 39.8 | 20 .6 | 69.4 | | |
| 13.7 | 60.4 | 22.2 | 32.8 | | |

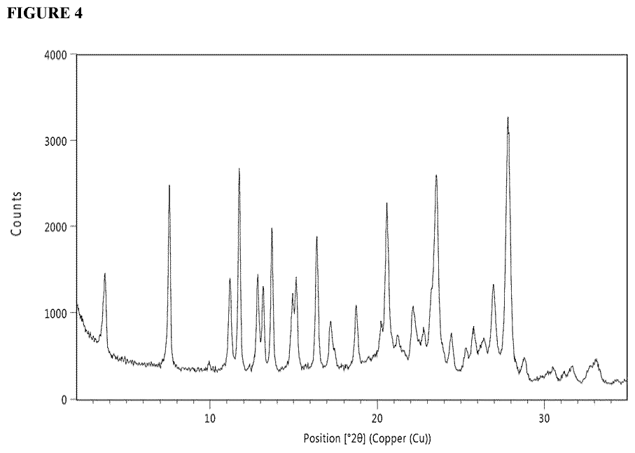

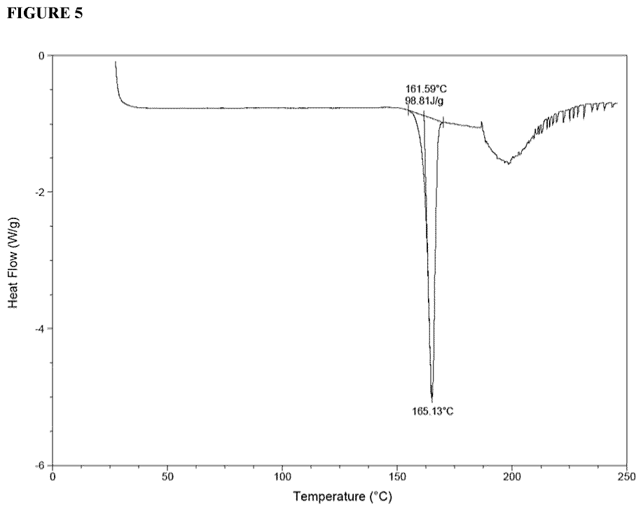

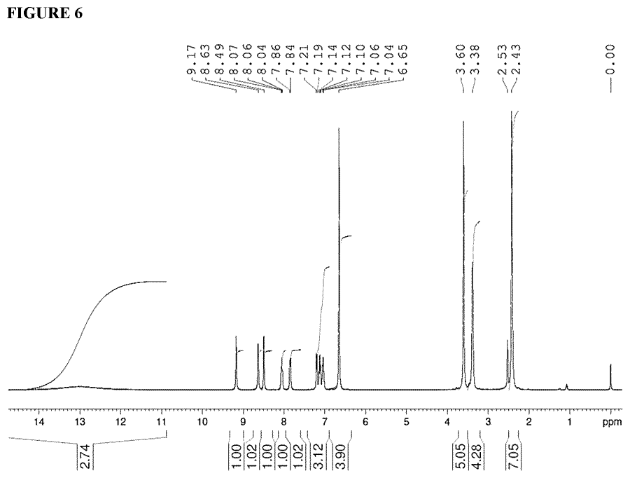

The solid form can be also characterized by XRPD pattern depicted in FIG. 4. The solid form can be further characterized by DSC pattern depicted in FIG. 5 and NMR spectrum depicted in FIG. 6.

The solid Form I can be prepared by a process comprising:
a. Dissolving of Omecamtiv mecarbil in ethanol;
b. Adding of fumaric acid;
c. Isolating the solid form.

The concentration of Omecamtiv mecarbil in ethanol can be between 0.04 and 0.1 g/ml. Omecamtiv mecarbil can be dissolved at an elevated temperature for example between 50° C. and 90° C., preferably between 60° C. and 85° C. To the solution fumaric acid is added. The molar ratio between Omecamtiv mecarbil and fumaric acid can be between 1:1 and 1:2, preferably it is 1:1.5. Fumaric acid can be added in solid form or in a form of a solution for example in the solvent used for dissolving of Omecamtiv mecarbil. In case that fumaric acid is used as a solution the concentration of the solution can be between 0.04 and 0.15 g/ml. The mixture is then cooled to temperature between −30° C. and 40° C., preferably between 0° C. and 30° C., more preferably between 15° C. and 25° C. The mixture can be optionally seeded with Omecamtiv mecarbil fumaric acid (1:2) salt, Form I. The mixture is then stirred for between 1 and 20 hours, preferably between 1 and 5 hours. In case no solid appears the mixture is cooled to temperature between −30° C. and −20° C. and stirred at this temperature for between and 30 hours. The solid form can be isolated by any suitable technique, for example using filtration or centrifuge.

The preparation of a solid form of Omecamtiv mecarbil fumaric acid (1:2) salt was tested also in following solvent: methanol, isopropanol, 1-butanol, 2-butanone and acetone. No solid form appeared.

Omecamtiv mecarbil fumaric acid (1:2) salt, Form I, can also be prepared by liquid-assisted grinding method utilizing Fritsch Pulverisette 23 vibratory mill. Omecamtiv mecarbil and fumaric acid are placed into zirconium oxide milling chamber with two milling balls of diameter 1 cm (BPR, ball-to powder ratio, approx. 40:1). The molar ratio between Omecamtiv mecarbil and fumaric acid can be between 1:2 and 1:2.2, preferably it is 1:2. To the mixture, small amount of solvent (for example tetrahydrofurane) is added. The concentration of Omecamtiv mecarbil in the solvent was 0.4 g/ml. The mixture is then oscillated during 90 min at 15 Hz. Solvent is freely evaporated and the crystalline product is isolated from the chamber.

The presented invention further relates to Omecamtiv mecarbil fumaric acid (1:1) salt, a solid form (Form I) thereof and a process for preparation thereof. The solid form, Form 1, can be characterized by XRPD pattern having 2θ values 6.7°, 13.0° and 15.8° degrees 2 theta (±0.2 degrees 2 theta). The solid Form 1 can be also characterized by XRPD pattern having 2θ values 6.7°, 11.0°, 13.0°, 15.8° and 17.0° degrees 2 theta (f 0.2 degrees 2 theta). The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 6.7 | 100.0 | 17.7 | 24.9 | 26.1 | 11.7 |
| 7.9 | 4.6 | 18.5 | 11.6 | 27.1 | 17.1 |
| 11.0 | 27.9 | 19.1 | 17.0 | 27.6 | 12.0 |
| 13.0 | 59.4 | 19.7 | 24.5 | 29.1 | 7.2 |
| 15.1 | 7.3 | 2θ .6 | 21.1 | 30.4 | 6.7 |
| 15.8 | 36.0 | 21.7 | 9.7 | 31.3 | 5.7 |
| 16.7 | 18.4 | 22.8 | 26.0 | 32.9 | 5.2 |
| 17.0 | 28.5 | 23.9 | 29.7 | 34.8 | 7.0 |

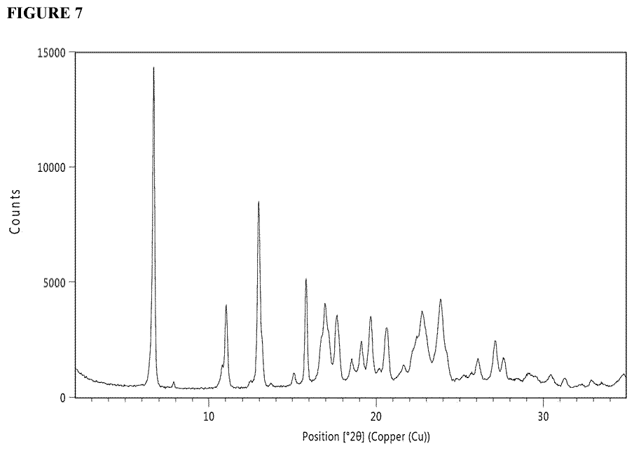

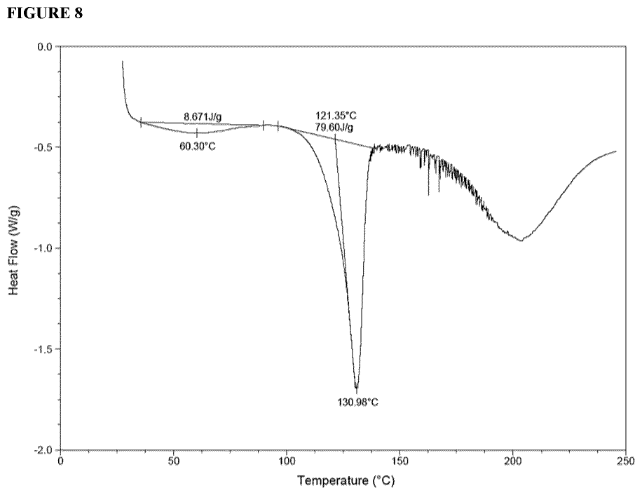

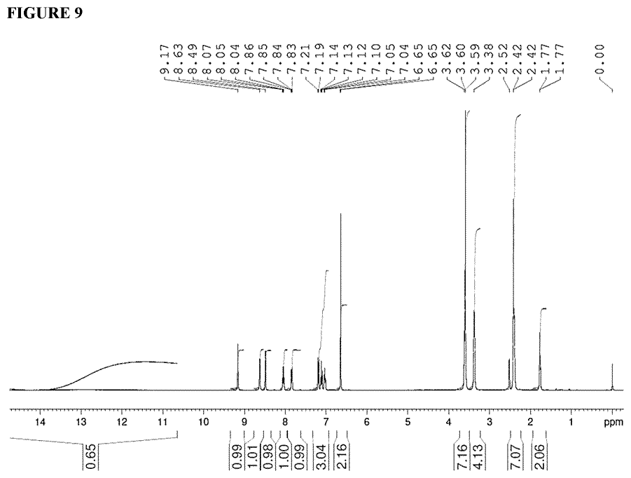

The solid Form 1 can be further characterized by XRPD pattern depicted in FIG. 7 and DSC pattern depicted in FIG. 8 and NMR spectrum depicted in FIG. 9. The solid Form 1 of Omecamtiv mecarbil fumaric acid (1:1) salt can be prepared by a process comprising:
a. Dissolving of Omecamtiv mecarbil in tetrahydrofurane;
b. Adding of fumaric acid;
c. Isolating the solid form.

The concentration of Omecamtiv mecarbil in the solvent can be 0.01 g/ml and 0.07 g/ml, preferably it is between 0.03 g/ml and 0.05 g/ml.

Omecamtiv mecarbil can be dissolved at an elevated temperature for example between is 50° C. and 80° C. To the solution fumaric acid is added. Fumaric acid can be added as solid or in form of a solution in a solvent for example in the solvent used for dissolving Omecamtiv mecarbil. In case that fumaric acid is used as a solution the concentration of the solution can be between 0.04 and 0.15 g/ml. The molar ration between Omecamtiv mecarbil and malonic acid can be between 1:1 and 1:1.3, preferably it is 1:1.

The mixture is cooled to a temperature between −30° C. and 40° C., preferably between 0° C. and 30° C., more preferably between 15° C. and 25° C. The mixture can be optionally seeded with Omecamtiv mecarbil fumaric acid (1:1) salt, Form I. The mixture is then stirred for between 1 and 20 hours, preferably between 1 and 5 hours. In case no solid appears the mixture is cooled to a temperature between −30° C. and −20° C. and stirred at this temperature for between 10 and 30 hours. The solid form can be isolated by any suitable technique, for example using filtration or centrifuge.

The preparation of a solid form of Omecamtiv mecarbil fumaric acid (1:1) salt was tested also in following solvent: methanol, ethanol, isopropanol, 1-butanol, 2-butanone and acetone. No solid form appeared.

When the Omecamtiv mecarbil fumaric acid (1:1), Form I, is kept at a temperature between 40° C. and 70° C. and humidity between 70% of relative humidity and 100% of relative humidity for between 20 and 40 days, it is transformed to Omecamtiv mecarbil fumaric acid (1:1), Form II. The Form II can be characterized by XRPD pattern having 2θ values 5.0°, 9.3° and 16.5° degrees 2 theta (f 0.2 degrees 2 theta). The solid Form II can be also characterized by XRPD pattern having 2θ values 5.0°, 9.3°, 16.5°, 18.6° and 24.6° degrees 2 theta (±0.2 degrees 2 theta). The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 5.0 | 100.0 | 19.4 | 19.6 | 27.0 | 5.8 |
| 8.9 | 3.6 | 20 .0 | 14.2 | 27.3 | 4.8 |
| 9.3 | 45.5 | 20 .2 | 24.3 | 27.8 | 19.2 |
| 9.9 | 11.7 | 20 .5 | 12.3 | 28.4 | 7.6 |
| 10.6 | 3.8 | 21.0 | 5.6 | 28.6 | 5.4 |
| 11.1 | 19.7 | 21.4 | 4.7 | 29.1 | 3.0 |
| 11.8 | 2.5 | 22.0 | 23.5 | 30.2 | 4.5 |
| 12.2 | 16.3 | 22.5 | 11.6 | 30.4 | 5.0 |
| 12.8 | 8.0 | 22.8 | 21.2 | 31.0 | 2.2 |
| 13.6 | 4.5 | 23.1 | 9.3 | 31.5 | 4.8 |
| 14.1 | 9.1 | 23.5 | 5.8 | 32.1 | 4.2 |
| 14.5 | 7.1 | 23.9 | 9.1 | 32.6 | 3.6 |
| 14.9 | 6.7 | 24.3 | 6.8 | 33.3 | 3.9 |
| 15.9 | 4.7 | 24.6 | 46.9 | 33.6 | 3.4 |
| 16.5 | 42.2 | 25.0 | 7.2 | 34.4 | 2.0 |
| 17.2 | 11.4 | 25.5 | 4.7 | 34.8 | 2.0 |
| 18.0 | 17.8 | 25.9 | 4.9 | | |
| 18.6 | 21.6 | 26.2 | 3.5 | | |

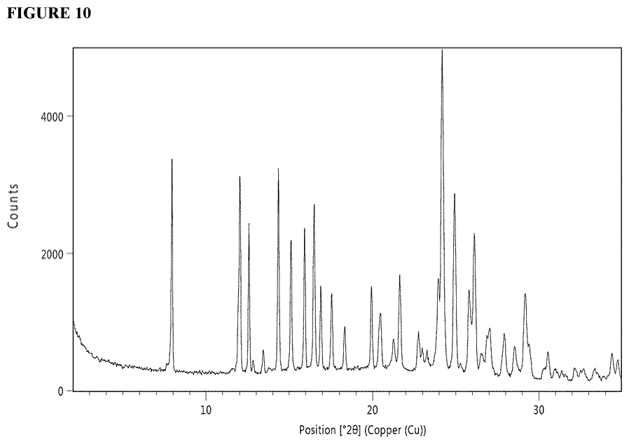

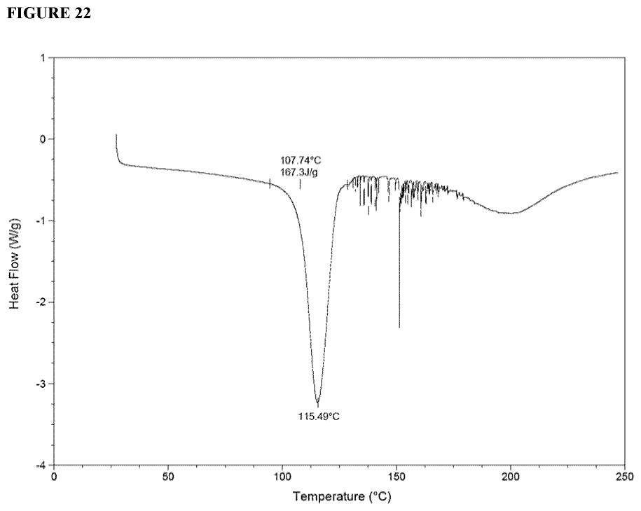

The solid Form II can be further characterized by XRPD pattern depicted in FIG. 10 and DSC pattern depicted in FIG. 22. The melting point of Form II is 116° C.

The presented invention further relates to Omecamtiv mecarbil malonic acid (1:1) acid salt, a solid form thereof and a process for preparation thereof.

The solid form, Form I, can be characterized by XRPD pattern having 2θ values 10.5°, 18.8° and 19.3° 2 theta (±0.2 degrees 2 theta). The solid Form I can be also characterized by XRPD pattern having 2θ values 10.5°, 18.8°, 19.3°, 22.9° and 23.7° degrees 2 theta (f 0.2 degrees 2 theta). The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 5.9 | 11.6 | 19.3 | 100.0 | 26.4 | 79.3 |
| 7.2 | 26.8 | 20 .2 | 22.2 | 27.1 | 9.4 |
| 7.8 | 9.1 | 20 .6 | 23.7 | 27.5 | 6.5 |
| 10.5 | 52.5 | 21.1 | 25.5 | 28.0 | 16.2 |
| 11.8 | 36.8 | 21.5 | 31.6 | 28.3 | 11.9 |
| 15.1 | 45.5 | 21.8 | 18.7 | 28.9 | 7.4 |
| 15.5 | 15.8 | 22.5 | 11.6 | 29.4 | 7.5 |
| 15.7 | 10.5 | 22.9 | 62.8 | 29.9 | 16.8 |
| 16.3 | 33.7 | 23.7 | 40.8 | 30.5 | 34.5 |
| 16.6 | 27.7 | 24.1 | 15.6 | 31.2 | 10.7 |
| 17.0 | 13.4 | 24.7 | 11.4 | 31.7 | 11.9 |
| 18.0 | 28.3 | 25.1 | 8.5 | 33.5 | 4.8 |
| 18.8 | 68.1 | 25.6 | 34.1 | | |

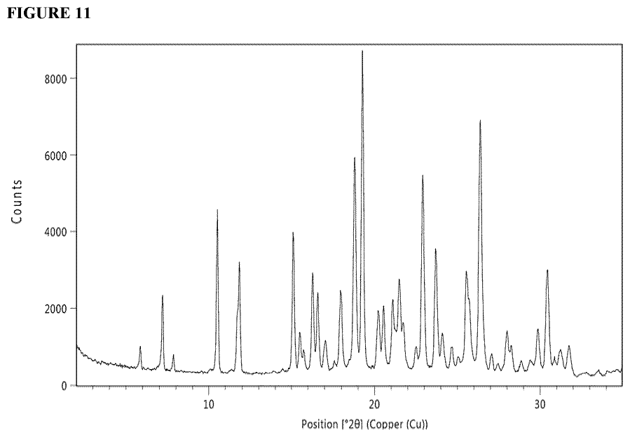

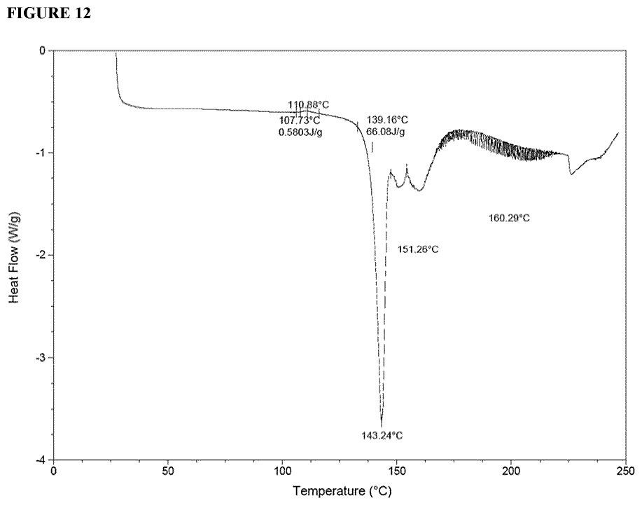

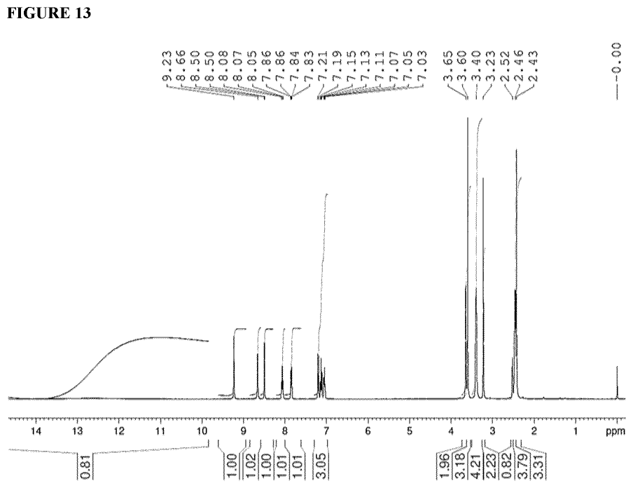

The solid Form I can be further characterized by XRPD pattern depicted in FIG. 11 and DSC pattern depicted in FIG. 12 and NMR spectrum depicted in FIG. 13. The solid form of Omecamtiv mecarbil malonic acid (1:1) salt can be prepared by a process comprising:
a. Dissolving of Omecamtiv mecarbil in tetrahydrofurane;
b. Adding of malonic acid;
c. Isolating the solid form.

The concentration of Omecamtiv mecarbil in tetrahydrofurane can be between 0.01 g/ml and 0.07 g/ml, preferably between 0.02 g/ml and 0.05 g/ml. Omecamtiv mecarbil can be dissolved at an elevated temperature for example between 40° C. and 80° C., preferably between 50° C. and 70° C. To the solution malonic acid is added. Malonic acid can be added as solid or in form of a solution in tetrahydrofurane. In case malonic acid is used as a solution the concentration of the solution can be between 0.07 g/ml and 0.2 g/ml, preferably between 0.1 g/ml and 0.15 g/ml. The molar ration between Omecamtiv mecarbil and malonic acid can be between 1:1 and 1:1.2, preferably it is 1:1. The mixture can be optionally seeded with Omecamtiv mecarbil malonic acid (1:1) salt, Form I.

The mixture is cooled to a temperature between −30° C. and 30° C., preferably between 20° C. and 30° C., and stirred for between 1 and 20 hours to obtain solid Form I. The solid form can be isolated by any suitable technique, for example using filtration or centrifuge.

The preparation of a solid form of Omecamtiv mecarbil malonic acid (1:1) salt was tested also in following solvent: methanol, ethanol, isopropanol, 1-butanol, 2-butanone and acetone. No solid form appeared.

Omecamtiv mecarbil malonic acid (1:1) salt, Form I, can also be prepared by liquid-assisted grinding method utilizing Fritsch Pulverisette 23 vibratory mill. Omecamtiv mecarbil and malonic acid are placed into zirconium oxide milling chamber with two milling balls of diameter 1 cm (BPR approx. 40:1). The molar ratio between Omecamtiv mecarbil and malonic acid can be between 1:1 and 1:1.2, preferably it is 1:1. To the mixture, small amount of solvent (for example tetrahydrofurane) was added. The concentration of Omecamtiv mecarbil in the solvent was 0.2 g/ml. Then the mixture is oscillated during 90 minutes at 15 Hz. Solvent is then freely evaporated and the crystalline product was isolated from the chamber.

The presented invention further relates to Omecamtiv mecarbil adipic acid (1:1.5) salt, a solid form (Form I) thereof and a process for preparation thereof. The solid form, Form I, can be characterized by XRPD pattern having 2θ values 4.9°, 9.3°, 16.5° and 24.6° degrees 2 theta (±0.2 degrees 2 theta). The solid Form I can be also characterized by XRPD pattern having 2θ values 4.9°, 9.3°, 16.5°, 22.1° and 24.6° degrees 2 theta (±0.2 degrees 2 theta).

The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 4.9 | 100.0 | 16.5 | 39.6 | 24.6 | 46.9 |
| 9.3 | 46.5 | 17.2 | 9.9 | 25.1 | 8.0 |
| 9.9 | 10.7 | 18.0 | 17.0 | 25.5 | 6.5 |
| 10.6 | 4.5 | 18.6 | 20 .5 | 25.9 | 5.6 |
| 11.1 | 18.2 | 19.4 | 17.1 | 27.1 | 6.3 |
| 12.2 | 15.8 | 19.9 | 14.2 | 27.8 | 18.8 |
| 12.7 | 7.1 | 20 .2 | 22.2 | 28.5 | 8.6 |
| 13.6 | 4.8 | 20 .6 | 15.2 | 30.2 | 4.8 |
| 14.1 | 8.7 | 22.1 | 26.7 | 31.4 | 4.8 |
| 14.5 | 7.1 | 22.8 | 22.1 | 32.2 | 5.1 |
| 14.9 | 7.2 | 23.1 | 10.6 | 33.3 | 4.3 |
| 15.9 | 4.8 | 23.9 | 10.3 | 33.5 | 4.2 |

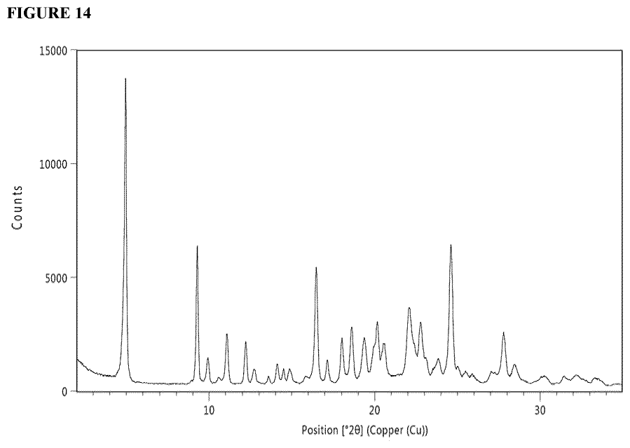

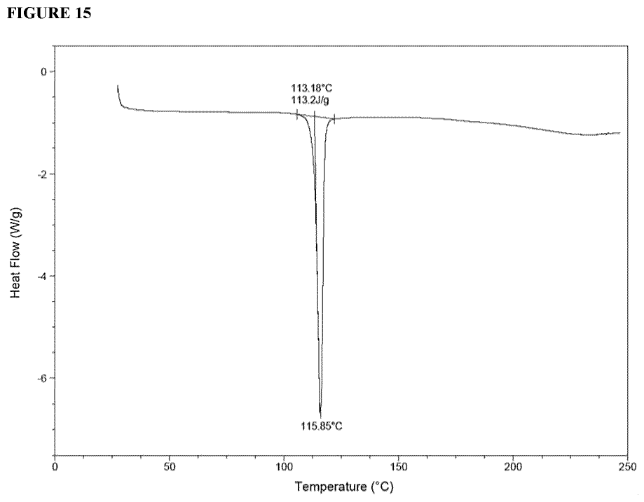

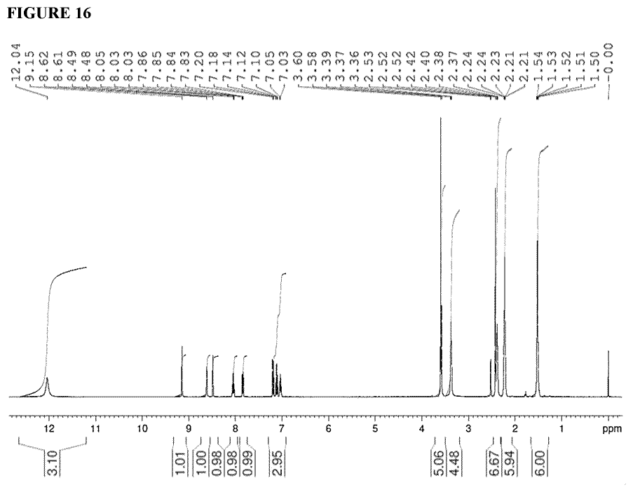

The solid Form I can be further characterized by XRPD pattern depicted in FIG. 14 and DSC pattern depicted in FIG. 15 and NMR spectrum depicted in FIG. 16. The solid form of Omecamtiv mecarbil adipic acid (1:1.5) salt can be prepared by a process comprising:
a. Dissolving of Omecamtiv mecarbil in tetrahydrofurane or acetone;
b. Adding of adipic acid;
c. Isolating the solid form.

The concentration of Omecamtiv mecarbil in tetrahydrofurane or acetone can be between 0.01 g/ml and 0.1 g/ml, preferably it is between 0.02 g/ml and 0.05 g/ml.

Omecamtiv mecarbil can be dissolved at an elevated temperature for example between 50° C. and 90° C., preferably between 60° C. and 70° C. To the solution adipic acid is added.

Adipic acid can be added as solid or in form of a solution in a solvent for example in the solvent used for dissolving Omecamtiv mecarbil. In case adipic acid is used as a solution the concentration of the solution can be between 0.04 and 0.15 g/ml. The molar ration between Omecamtiv mecarbil and adipic acid can be between 1:1.8 and 1:2.3, preferably it is 1:2.

The mixture is cooled to a temperature between −30° C. and 40° C., preferably between 0° C. and 30° C., more preferably between 15° C. and 25° C. The mixture can be optionally seeded with Omecamtiv mecarbil adipic acid (1:1.5) salt, Form I. The mixture is then stirred for between 1 and 20 hours, preferably between 1 and 5 hours. In case no solid appears the mixture is cooled to a temperature between −30° C. and −20° C. and stirred at this temperature for between 1 and 30 hours to provide the solid Form I. The solid form can be isolated by any suitable technique, for example using filtration or centrifuge.

The preparation of a solid form of Omecamtiv mecarbil adipic acid (1:1.5) salt was tested also in following solvent: methanol, ethanol, isopropanol, 1-butanol or 2-butanone. No solid form appeared.

Omecamtiv mecarbil adipic acid (1:1.5) salt, Form I can also be prepared by liquid-assisted grinding method utilizing Fritsch Pulverisette 23 vibratory mill. Omecamtiv mecarbil and adipic acid are placed into zirconium oxide milling chamber with two milling balls of diameter 1 cm (BPR approx. 40:1). The molar ratio between Omecamtiv mecarbil and adipic acid can be between 1:1.8 and 1:2.3, preferably it is 1:2. To the mixture, small amount of solvent (for example tetrahydrofurane) is added. The concentration of Omecamtiv mecarbil in the solvent was 0.2 g/ml. Then, the mixture is oscillated during 90 minutes at 15 Hz. Solvent is then freely evaporated and the crystalline product, Form I, is isolated from the chamber.

The presented invention further relates to Omecamtiv mecarbil sulfuric acid salt, solid forms (Form I and Form II and Form III) thereof and processes for preparation thereof.

The solid Form I can be characterized by XRPD pattern having 2θ values 7.30, 13.0, 14.60 and 20.40 degrees 2 theta (f 0.2 degrees 2 theta). The solid Form I can be also characterized by XRPD pattern having 2θ values 7.30, 13.0, 14.60, 16.40, 16.90 and 20.40 degrees 2 theta (±0.2 degrees 2 theta). The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 7.3 | 90.6 | 18.3 | 10.3 | 24.5 | 17.1 |
| 8.0 | 22.1 | 18.8 | 26.7 | 25.1 | 23.0 |
| 11.7 | 15.3 | 19.6 | 10.4 | 25.5 | 13.1 |
| 12.2 | 19.0 | 20.4 | 100.0 | 25.9 | 17.4 |
| 12.5 | 11.6 | 21.0 | 29.7 | 26.6 | 39.5 |
| 13.6 | 86.1 | 21.4 | 17.8 | 27.3 | 44.6 |
| 14.6 | 69.5 | 21.8 | 24.8 | 28.1 | 12.8 |
| 14.9 | 17.0 | 22.0 | 30.8 | 29.0 | 12.8 |
| 16.1 | 19.4 | 22.3 | 15.0 | 29.5 | 15.0 |
| 16.3 | 43.5 | 22.9 | 55.1 | 30.0 | 6.7 |
| 16.4 | 50.7 | 23.5 | 14.5 | 32.5 | 6.3 |
| 16.9 | 29.5 | 23.8 | 39.5 | 33.1 | 8.9 |
| 17.4 | 18.5 | 24.2 | 41.1 | 33.8 | 9.7 |

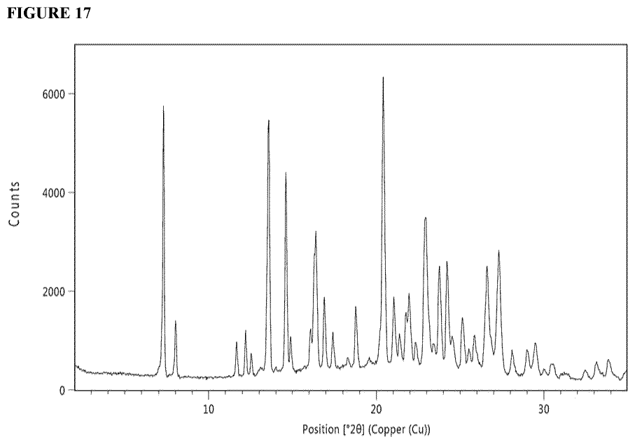

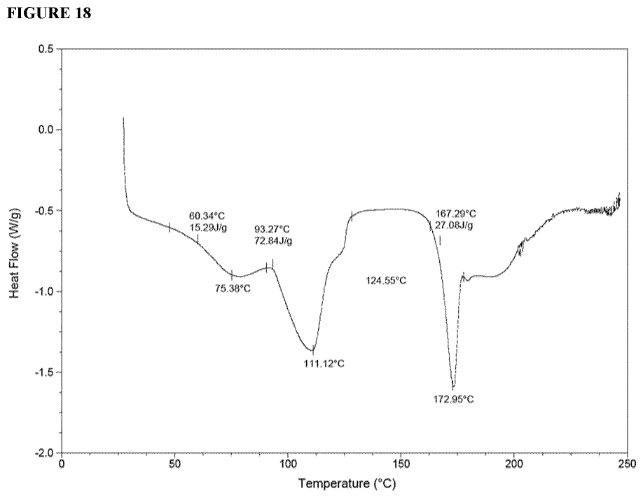

The solid Form I can be further characterized by XRPD pattern depicted in FIG. 17 and DSC pattern depicted in FIG. 18. The solid Form I of Omecamtiv mecarbil sulfuric acid salt can be prepared by a process comprising:
a. Dissolving of Omecamtiv mecarbil in tetrahydrofurane;
b. Adding of sulfuric acid;
c. Isolating the solid form.

The concentration of Omecamtiv mecarbil in tetrahydrofurane can be 0.01 g/ml and 0.1 g/ml, preferably it is between 0.02 g/ml and 0.05 g/ml.

Omecamtiv mecarbil can be dissolved at an elevated temperature for example between 50° C. and 90° C., preferably between 60° C. and 70° C. To the solution sulfuric acid is added, preferably dropwise. Sulfuric acid is preferably used as 98% solution (concentrated sulfuric acid). The molar ration between Omecamtiv mecarbil and sulfuric acid can be between 1:1.8 and 1:2.3, preferably it is 1:2. The mixture is stirred at the elevated temperature for between 12 and 24 hours. The mixture was filtered off to provide Omecamtiv mecarbil sulfuric acid is salt Form I. The solid form can be isolated by any suitable technique, for example using filtration or centrifuge.

The preparation of a solid form of Omecamtiv mecarbil sulfuric acid salt was tested also in following solvent: methanol, ethanol, isopropanol, 1-butanol, acetone or 2-butanone. No solid form appeared.

The invention also relates to solid Form II of Omecamtiv mecarbil sulfuric acid salt that can be characterized by XRPD pattern having 2θ values 5.1°, 7.7° and 14.3° degrees 2 theta (±0.2 degrees 2 theta). The solid Form II can be also characterized by XRPD pattern having 2θ values 7.3°, 13.60, 14.60, 16.4°, 16.9° and 20.4° degrees 2 theta (±0.2 degrees 2 theta). The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 5.1 | 79.2 | 17.4 | 34.3 | 23.5 | 36.1 |
| 7.7 | 100.0 | 17.6 | 31.5 | 24.5 | 71.7 |
| 10.2 | 11.2 | 18.3 | 25.3 | 26.3 | 42.6 |
| 13.9 | 20.4 | 18.6 | 85.4 | 26.6 | 45.3 |
| 14.2 | 31.1 | 20 .3 | 55.5 | 27.2 | 45.3 |
| 14.5 | 86.1 | 20 .5 | 57.5 | 27.9 | 49.2 |
| 14.9 | 30.2 | 21.4 | 31.0 | 28.6 | 29.1 |
| 15.5 | 40.2 | 22.2 | 58.5 | 29.0 | 33.9 |
| 16.2 | 26.8 | 22.4 | 83.0 | 29.4 | 24.8 |
| 16.5 | 72.5 | 22.8 | 36.7 | 31.4 | 14.5 |

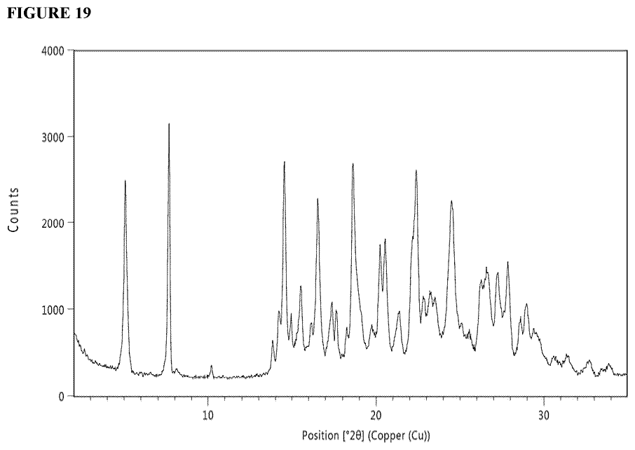

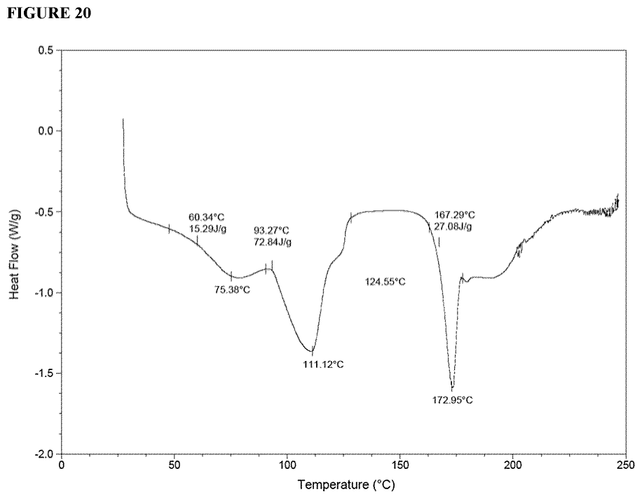

The solid Form II can be further characterized by XRPD pattern depicted in FIG. 19 and DSC pattern depicted in FIG. 20. The solid Form II of Omecamtiv mecarbil sulfuric acid salt can be prepared by a process comprising drying a solid form of Omecamtiv mecarbil sulfuric acid salt, preferably solid Form I, at 50° C. for between 12 and 24 hours.

When the Omecamtiv mecarbil sulfuric acid salt, Form II, is kept at a temperature between 40° C. and 80° C. and humidity between 70% of relative humidity and 100% of relative humidity for between 20 and 40 days, it is transformed to Omecamtiv mecarbil sulfuric acid salt, Form III. The Form III can be characterized by XRPD pattern having 20 values 14.3°, 19.3° and 25.8° degrees 2 theta (±0.2 degrees 2 theta). The solid Form II can be also characterized by XRPD pattern having 2θ values 5.4°, 7.5°, 14.3°, 19.3° and 25.8° degrees 2 theta (±0.2 degrees 2 theta). The solid form can be further characterized by XRPD pattern described in the following table:

| Angle | Intensity | Angle | Intensity | Angle | Intensity |
|---|---|---|---|---|---|
| (2θ ) | (%) | (2θ ) | (%) | (2θ ) | (%) |
| 5.4 | 27.7 | 17.7 | 15.6 | 26.5 | 14.7 |
| 7.5 | 26.2 | 18.6 | 19.9 | 27.2 | 8.0 |
| 8.3 | 12.9 | 19.3 | 100.0 | 27.7 | 29.9 |
| 10.1 | 7.0 | 19.8 | 13.4 | 28.1 | 22.0 |
| 11.9 | 6.7 | 20.2 | 26.1 | 29.0 | 10.9 |
| 14.0 | 8.5 | 20.9 | 26.9 | 29.2 | 11.3 |
| 14.3 | 69.9 | 21.4 | 34.6 | 30.0 | 20.9 |
| 14.6 | 11.9 | 22.2 | 37.6 | 30.6 | 8.2 |
| 15.1 | 13.7 | 22.8 | 15.9 | 31.5 | 5.7 |
| 15.6 | 13.0 | 23.1 | 42.9 | 32.2 | 7.9 |
| 16.0 | 14.9 | 23.5 | 18.9 | 32.6 | 5.3 |
| 16.2 | 20.8 | 23.8 | 31.7 | 33.1 | 4.9 |
| 16.4 | 13.3 | 24.4 | 36.7 | 33.8 | 5.9 |
| 16.7 | 14.8 | 25.1 | 35.4 | 34.2 | 4.4 |
| 17.0 | 41.3 | 25.8 | 46.3 | 34.7 | 4.5 |

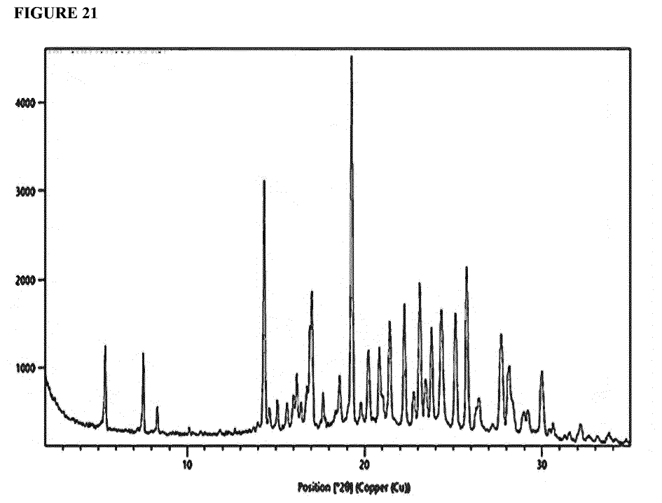

The solid Form III can be further characterized by XRPD pattern depicted in FIG. 21.

The salt of presented invention can be used in a pharmaceutical composition for the treatment of conditions treatable by Omecamtiv mecarbil.

The invention will be further described with reference to the following examples.

### EXAMPLES

Nuclear magnetic resonance spectroscopy (NMR) was performed using Avance III 400 MHz NMR spectrometer.

DCS patterns were obtained using the following conditions: 10° C./min->250° C.

XRPD spectrum was obtained using the following measurement conditions:

Panalytical Empyrean diffractometer with Θ/2Θ geometry (transmition mode), equipped with a PixCell 3D detector;

| | |
|---|---|
| Start angle (2θ): | 2.0° |
| End angle (2θ): | 35.0° |
| Step size: | 0.026° |
| Scan speed: | 0.0955°/seconds |
| Radiation type: | Cu |
| Radiation wavelengths: | 1.5406Å (Kα1), primary monochromator used |
| Divergence slit: | 1/2° |
| Antiscatter slit: | 1/2° |
| Soller slit: | 0.02 rad |
| Detector slit: | 7.5 mm |
| Rotation speed: | 30 rpm |

### Example 1: Omecamtiv Mecarbil Salt with Maleic Acid, Form A

250 mg of Omecamtiv mecarbil was dissolved in a solvent (in Table below) at a temperature (in Table below). 72.3 mg of maleic acid (1 eq. w.r.t. Omecamtiv mecarbil) in 1 ml of the solvent was added. The mixture was cooled to 25° C. and stirred for 16 hours. Solid mass was filtered off and dried. XRPD of prepared Form A is depicted in FIG. 1, DSC is depicted in FIG. 2, NMR is depicted in FIG. 3. The ratio between Omecamtiv mecarbil and the salt was determined by NMR as 1:2 (i.e. dimaleate salt).

| Solvent | Volume of solvent | Temperature | Results |
|---|---|---|---|
| Methanol | 3.5 ml | 65° C. | Crystalline form A |
| Ethanol | 2.5 ml | 78° C. | Crystalline form A |
| Isopropanol | 5.0 ml | 82° C. | Crystalline form A |
| 1-Butanol | 3.0 ml | 100° C. | Crystalline form A |
| Acetone | 200 ml | 56° C. | Crystalline form A |
| 2-Butanone | 10 ml | 80° C. | Crystalline form A |
| Tetrahydrofurane | 6.5 ml | 66° C. | Crystalline form A |

### Example 2: Omecamtiv Mecarbil Fumaric Acid (1:2) Salt, Form I

1 g of Omecamtiv mecarbil was dissolved in 14 ml of ethanol at 68° C. Then 0.579 g of fumaric acid in 8 ml of hot (68° C.) ethanol was added. The mixture was spontaneously cooled to 25° C. and the mixture was stirred for 1 hour at 25° C. The mixture was then placed into a freezer at −24° C. for 24 hours to obtain solid Form I. The solid Form I was dried under vacuum (100 mbar, N₂ bleed) at 50° C. for 24 hours to obtain 0.77 g (65.3% of theoretical yield) of Omecamtiv mecarbil fumaric acid (1:2) salt, Form I. XRPD of obtained solid corresponds to XRPD pattern depicted in FIG. 4. DSC pattern of obtained solid is depicted in FIG. 5 and NMR spectrum of obtained solid is depicted in FIG. 6. Melting point 163.8° C.

The solid Form I of Omecamtiv mecarbil fumaric acid (1:2) salt can be also obtain by liquid-assisted grinding method utilizing Fritsch Pulverisette 23 vibratory mill. 100 mg of Omecamtiv mecarbil and 57.8 mg of fumaric acid were placed into zirconium oxide milling chamber with two milling balls of diameter 1 cm (BPR (ball-to-powder ratio) approx. 40:1). To the mixture, 0.25 ml of tetrahydrofurane was added. Then, the mixture was oscillated during 90 min at 15 Hz. Solvent was then freely evaporated and the crystalline product was isolated from the chamber. The XRPD pattern of obtained solid product corresponds to XRPD pattern depicted in FIG. 4. DSC pattern of obtained solid is depicted in FIG. 5 and NMR spectrum of obtained solid is depicted in FIG. 6. Melting point 163.8° C.

### Example 3: Omecamtiv Mecarbil Fumaric Acid (1:1) Salt, Form I

1 g of Omecamtiv mecarbil was dissolved in 26 ml of tetrahydrofurane at 65° C. 0.578 g of fumaric acid in 10 ml of hot (65° C.) tetrahydrofurane and the mixture was spontaneously cooled to 25° C. The mixture was stirred for 1 hour at 25° C. and then placed into a freezer at −25° C. Crystallization occurred spontaneously after 24 hours. The solid that crystallized was filtered, dried under vacuum (100 mbar, N₂ bleed) at 50° C. for 24 hours to obtain 1.23 g (89% of the theoretical yield) of Omecamtiv mecarbil fumaric acid (1:1) salt, Form I. XRPD of obtained solid corresponds to XRPD pattern depicted in FIG. 7. DSC pattern of obtained solid is depicted in FIG. 8 and NMR spectrum of obtained solid is depicted in FIG. 9. Melting point 134° C.

### Example 4: Omecamtiv Mecarbil Malonic Acid (1:1) Salt, Form I

1 g of Omecamtiv mecarbil was dissolved in 26 ml of tetrahydrofurane at 65° C. 0.259 g of malonic acid in 2 ml of hot (65° C.) tetrahydrofurane was added. The mixture was spontaneously cooled to 25° C. and stirred at this temperature for 1 hour. The solid was filtered, dried under vacuum (100 mbar, N₂ bleed) at 50° C. for 24 hours to obtain 0.9 g (71% of the theoretical yield) of Omecamtiv mecarbil malonic acid (1:1) salt, Form I. XRPD of the solid corresponds to the XRPD pattern depicted in FIG. 11. DSC pattern of obtained solid is depicted in FIG. 12 and NMR spectrum of obtained solid is depicted in FIG. 13. Melting point 153.9° C.

### Example 5: Omecamtiv Mecarbil Adipic Acid (1:1.5) Salt, Form I

1 g of Omecamtiv mecarbil was dissolved in 26 ml of tetrahydrofurane at 65° C. 0.578 g of fumaric acid in 10 ml of hot (65° C.) tetrahydrofurane was added, the mixture was spontaneously cooled to 25° C. The mixture was stirred for 1 hour at 25° C. and it was placed into a freezer for 2 hours. The solid was filtered, dried under vacuum (100 mbar, N₂ bleed) at 50° C. for 24 hours to obtain 1.15 g of (75% of the theoretical yield) of Omecamtiv mecarbil adipic acid (1:1.5) salt, Form I. XRPD of obtained solid corresponds to XRPD pattern depicted in FIG. 14. DSC pattern of obtained solid is depicted in FIG. 15 and NMR spectrum of obtained solid is depicted in FIG. 16. Melting point 116° C. Omecamtiv mecarbil adipic acid (1:1.5) salt, Form I can be also prepared by following procedure:

1 g of Omecamtiv mecarbil was dissolved in 26 ml of acetone at 65° C. 0.578 g of fumaric acid in 10 ml of hot (65° C.) acetone was added, the mixture was spontaneously cooled to 25° C. The resulting clear solution was seeded with Omecamtiv mecarbil adipic acid (1:1.5) salt, Form I. The mixture was stirred for 1 hour at 25° C. and it was placed into a freezer for 2 hours. The solid was filtered, dried under vacuum (100 mbar, N₂ bleed) at 50° C. for 24 hours to obtain 1.15 g of (75% of the theoretical yield) of Omecamtiv mecarbil adipic acid (1:1.5) salt, Form I. XRPD of obtained solid corresponds to XRPD pattern depicted in FIG. 14. DSC pattern of obtained solid is depicted in FIG. 15 and NMR spectrum of obtained solid is depicted in FIG. 16. Melting point 116° C.

Omecamtiv mecarbil adipic acid (1:1.5) salt, Form I can be also prepared by liquid-assisted grinding method utilizing Fritsch Pulverisette 23 vibratory mill. 0.1 g of Omecamtiv mecarbil and 0.0728 g of adipic acid were placed into zirconium oxide milling chamber with two milling balls of diameter 1 cm (BPR approx. 40:1). To the mixture, 0.1 ml of THF was added. The mixture was oscillated during 90 minutes at 15 Hz. Solvent was then freely evaporated and the crystalline product was isolated from the chamber. XRPD of obtained solid corresponds to XRPD pattern depicted in FIG. 14. DSC pattern of obtained solid is depicted in FIG. 15 and NMR spectrum of obtained solid is depicted in FIG. 16. Melting point 116° C.

### Example 6: Omecamtiv Mecarbil Sulfuric Acid Salt, Form I

1 g of Omecamtiv mecarbil was dissolved in 26 ml of tetrahydrofurane at 65° C. 0.267 ml of sulfuric acid was added dropwise into the hot solution. The solid that formed upon addition of acid was stirred at 65° C. overnight. The white product was filtered off to obtain Omecamtiv mecarbil sulfuric acid salt, Form I. XRPD of obtained solid corresponds to XRPD pattern depicted in FIG. 17. DSC pattern of obtained solid is depicted in FIG. 18.

### Example 7: Omecamtiv Mecarbil Sulfuric Acid Salt, Form II

Omecamtiv mecarbil sulfuric acid salt, Form I was dried at 25-30° C. and 30-40% RH (relative humidity) for 60 hours to obtain 1.2 g (75% of the theoretical yield) of Omecamtiv mecarbil sulfuric acid salt, Form II.

XRPD of obtained solid corresponds to XRPD pattern depicted in FIG. 19. DSC pattern of obtained solid is depicted in FIG. 20.

### Example 7: Solubility of Omecamtiv Mecarbil Salts

The solubility of prepared salts was tested in water solution at different pH (1.2, 4.5 and 6.8) and compared to Omecamtiv mecarbil dihydrochloride monohydrate salt prepared according to a procedure disclosed in WO2006009726 application. The results are summarized in the following table. It can be concluded that solubilities of Omecamtiv mecarbil maleic acid salt, Form A, Omecamtiv mecarbil fumaric acid (1:1), Form I, Omecamtiv mecarbil malonic acid (1:1), Form I, Omecamtiv mecarbil adipic acid (1:1.5), Form I, Omecamtiv mecarbil sulfuric acid (1:1), Forms I, II and III salts are lower in comparison with Omecamtiv mecarbil dihydrochloride monohydrate salt. The solubility of Omecamtiv mecarbil fumaric acid (1:2), Form I is comparable to Omecamtiv mecarbil dihydrochloride monohydrate salt.

Omecamtiv Mecarbil salts-Category of solubility (Descriptive Term)¹

| | Solvent (aqueous buffer) | | |
|---|---|---|---|
| Salt | pH 1.2 | pH 4.5 | pH 6.8 |
| Salt with HC1 | Freely soluble | Freely soluble | Freely soluble |
| | (100-1000 mg/mL) | (100-1000 mg/mL) | (100-1000 mg/mL) |
| Salt with maleic acid, | Sparingly soluble | Sparingly soluble | Very slightly soluble |
| Form A | (10-33 mg/mL) | (10-33 mg/mL) | (0.1-1 mg/mL) |
| Salt with fumaric | Very slightly soluble | Sparingly soluble | Sparingly soluble |
| acid (1:1), Form I | | | |
| Salt with malonic | Sparingly soluble | Sparingly soluble | Very slightly soluble |
| acid (1:1), Form I | | | |
| Salt with adipic acid | Sparingly soluble | Sparingly soluble | Very slightly soluble |
| (1:1.5), Form I | | | |
| Salt with sulfuric | Sparingly soluble | Sparingly soluble | Sparingly soluble |
| acid (1:1), Forms I, II | | | |
| and III | | | |
| Salt with fumaric | Freely soluble | Freely soluble | Freely soluble |
| acid (1:2), Form I | | | |

¹According to the USP, section Reference Tables-Description and Solubility, corresponds with the Ph. Eur., Chapter 1. General Notices, section 1.4 Monographs-Solubility

1. Omecamtiv mecarbil malonic acid (1:1) salt.

2. A solid form of Omecamtiv mecarbil malonic acid (1:1) salt.

3. The solid form according to claim 2, wherein said solid form is crystalline Form I, characterized by XRPD pattern having 2θ values 10.5°, 18.8° and 19.3° degrees 2 theta (±0.2 degrees 2 theta).

4. The solid form according to claim 2 characterized by XRPD pattern having 2θ values 10.5°, 18.8°, 19.3°, 22.9° and 23.7° degrees 2 theta (±0.2 degrees 2 theta).

5. A process for preparation of the solid form according to claim 3 comprising:
a) dissolving of Omecamtiv mecarbil in tetrahydrofurane;
b) adding of malonic acid; and
c) Isolating the solid form.

6. Omecamtiv mecarbil adipic acid (1:1.5) salt.

7. A solid form of Omecamtiv adipic acid (1:1.5) salt.

8. The solid form according to claim 7, wherein said solid form is crystalline Form I, characterized by XRPD pattern having 2θ values 4.9°, 9.3°, 16.5° and 24.6° degrees 2 theta (±0.2 degrees 2 theta).

9. The solid form according to claim 7 characterized by XRPD pattern having 2θ values 4.9°, 9.3°, 16.5°, 22.1° and 24.6° degrees 2 theta (±0.2 degrees 2 theta).

10. A process for preparation of the solid form according to claim 8 comprising:
a) dissolving of Omecamtiv mecarbil in tetrahydrofurane or acetone;
b) adding of adipic acid; and
c) isolating the solid form.

11. A solid form of Omecamtiv mecarbil fumaric acid (1:2) salt, wherein said solid Omecamtiv mecarbil fumaric acid salt is crystalline Form I, characterized by XRPD pattern having 2θ values 7.6°, 11.7°, 13.7° and 27.8° degrees 2 theta (±0.2 degrees 2 theta).

12. The solid form according to claim 11 characterized by XRPD pattern having 2θ values 7.6°, 11.2°, 11.7°, 13.7°, 16.4°, 20.6°, 23.5° and 27.8° degrees 2 theta (±0.2 degrees 2 theta).

13. A process for preparation of the solid form according to claim 11 comprising:
a) dissolving of Omecamtiv mecarbil in ethanol;
b) adding of fumaric acid; and
c) isolating the solid form.

14. A solid form of Omecamtiv mecarbil fumaric acid (1:1) salt, wherein said solid Omecamtiv mecarbil fumaric acid salt is crystalline Form 1, characterized by XRPD pattern having 2θ values 6.7°, 13.0° and 15.8° degrees 2 theta (+0.2 degrees 2 theta).

15. The solid form according to claim 14 characterized by XRPD pattern having 2θ values 6.7°, 11.0°, 13.0°, 15.8° and 17.0° degrees 2 theta (±0.2 degrees 2 theta).

16. A process for preparation of the solid form according to claim 14 comprising:
a) dissolving of Omecamtiv mecarbil in tetrahydrofurane;
b) adding of fumaric acid; and
c) isolating the solid form.

17. A solid form of Omecamtiv mecarbil sulfuric acid (1:1) salt, wherein said solid Omecamtiv mecarbil sulfuric acid salt is crystalline Form II, characterized by XRPD pattern having 2θ values 5.1°, 7.7° and 14.3° degrees 2 theta (±0.2 degrees 2 theta).

18. The solid form according to claim 17 characterized by XRPD pattern having 2θ values 5.1°, 7.7°, 14.3°, 15.5°, 16.5° and 18.6° degrees 2 theta (±0.2 degrees 2 theta).

19. A process for preparation of the solid form according to claim 17 comprising drying a solid form of Omecamtiv mecarbil sulfuric acid (1:1) salt at 50° C. for between 12 and 24 hours.

20. A pharmaceutical composition comprising the Omecamtiv mecarbil salt according to claim 1.
